(19) United States
(12) Patent Application Publication
(10) Pub. No.: US 2010/0196901 A1
(43) Pub. Date: Aug. 5, 2010

(54) Method for Obtaining Information on Biological Rhythm by Using Hair

(76) Inventors: Takuro Yamamoto, Kanagawa (JP); Makoto Akashi, Yamaguchi (JP); Shiko Yamashita, Tokyo (JP); Tomoteru Abe, Tokyo (JP); Kazuhiro Nakagawa, Saitama (JP); Haruhiko Soma, Tokyo (JP); Koichi Node, Saga (JP); Noriyuki Kishii, Kanagawa (JP); Tomohiro Hayakawa, Saitama (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/669,153

(22) PCT Filed: Jul. 16, 2008
(86) PCT No.: PCT/JP2008/062808

§ 371 Date: Jan. 14, 2010

(30) Foreign Application Priority Data
Jul. 25, 2007 (JP) 2007 193477

(51) Int. Cl. C12Q 1/68

(52) U.S. Cl. 435/6

(57) ABSTRACT

To provide a simple and low-invasive method for obtaining information on a biorhythm of an individual organism. Provided is a method for obtaining information on a biorhythm of an individual organism based on variations over time in an expression level of a clock gene in a hair follicle cell of the individual organism. According to this method, a phase shift of the biorhythm of the individual organism can be detected by preparing a plurality of times about the individual organism and conducting a collation. Further, a phase shift of the biorhythm of the individual organism can also be detected by collating an expression level of the clock gene at a predetermined time with the molecular clock table.

### TECHNICAL FIELD

This invention relates to a method for obtaining information on a biorhythm. More specifically, the present invention is concerned with a method for obtaining information especially on a circadian rhythm based on variations over time in an expression level of a clock gene in a hair follicle cell.

### BACKGROUND ART

Various biological responses (which can also be called “physiological processes”) in an individual organism are each known to show a rhythm that self-sustainably fluctuates (oscillates) in cycle. This rhythm is called “biorhythm.” In particular, a “circadian rhythm” which has a periodicity of about a day is considered to widely govern biological responses (physiological processes) such as sleep-wake cycles and daily fluctuations in body temperature, blood pressure and hormonal secretions. A circadian rhythm has also been revealed to also take part in daily fluctuations in mental and physical activities, locomotor ability and drug sensitivity.

A regulatory center (which is also called “central clock”) for a biorhythm resides in the suprachiasmatic nucleus of the hypothalamus. In each peripheral tissue, an independently-oscillating “peripheral clock” also resides. As several circadian rhythm cycles can also be detected in cultured cells, the function of a peripheral clock is considered to be found in each individual cell. It is considered that, while the peripheral clocks in each tissue or cells independently oscillate, these oscillations are synchronized and unified under the influence of the central clock, thereby expressing and regulating a biorhythm.

In the regulation of a biorhythm, a class of genes called “clock genes” take part. These clock genes function as “biological clocks” by causing their expression to fluctuate (oscillate) autonomically. The clock genes govern various biological responses (physiological processes), such as those described above, by regulating the expression of various genes in other classes.

A polymorphism or variation in clock genes has been revealed to become a cause of onset of a cancer, diabetes, vascular disease, neurodegenerative disease, or the like. In recent years, the involvement of a polymorphism or variation in clock genes has also been indicated as a cause of onset with respect to neurodegenerative diseases such as bipolar disorder and depression, and a treatment method has been used in an attempt to reset through exposure to light a biological clock modulated by a polymorphism or variation.

On the other hand, for example, sleep-wake cycles are subjected not only to autonomic regulations by biological clocks but also to restrictions through life in society. By a variation in daily bedtime or wake-up time, a rhythm shift (phase shift) may take place between “a sleep-wake cycle in real life” and “a sleep-wake cycle by biological clocks.” Such a rhythm shift is considered to become a cause of a so-called “jet lag” or a sleep disorder, or of such a mental disease as described above.

Further, the use of a biorhythm has also begun in an attempt to maximize the effects of drug treatment. Due to a circadian rhythm of a molecule as a target of a drug (drug target molecule) or an enzyme (drug-metabolizing enzyme) that metabolizes, the treatment effect of the drug is also considered to undergo daily fluctuations. A concept of “chronotherapy” has, therefore, been proposed that attempts to maximize the effect of treatment by setting an optimal dosage time for each drug.

As more familiar subjects of research, the use of the daily rhythms of mental or physical activities or locomotor ability has begun to study the time of activity that draws out one's ability to the maximum in learning or training and the time of meal ingestion that readily (or hardly) causes a weight gain.

From the foregoing, the accurate ascertainment of a biorhythm governed by biological clocks is believed to be very beneficial for the prevention of various diseases, the improvement of poor physical conditions such as time lag, the realization of chronotherapy, the exhibition of one's own ability, dieting, and the like.

PCT Patent Publication No. WO2004/012128 discloses a method for estimating a biological clock based on assay data of gene expression product levels in a standard sample collected from an individual organism. According to this biological clock estimation method, a molecular clock table for the estimation of the biological clock is prepared based on expression levels of the gene expression product level (specifically, mRNA). It is, however, to be noted that no specific collected tissue (or cells) or assay target gene is described in PCT Patent Publication No. WO2004/012128.

BMC Molecular Biology 5:18 (2004 Oct. 9) describes that in an experiment using mice, circadian rhythms of clock genes were detected in peripheral tissues of the heart, lungs, liver, stomach, spleen, kidneys, testicles and the like. It is, however, to be noted that no discussion is made on hair follicle cells in BMC Molecular Biology 5:18 (2004 Oct. 9).

American Journal of Pathology 158(5):1793-801 (2001 May) describes that circadian rhythms of clock genes were detected in the skin or tunica mucosa oris collected from human subjects.

PLoS Biology 3(10):e338 (2005 October, Epub 2005 Sep. 27) describes the results of an expression analysis of clock genes in fibroblasts by culturing and proliferating fibroblasts collected by skin biopsy from human subjects and introducing a reporter gene fused with a clock gene promoter. Further, American Journal of Pathology 158(5):1793-801 (2001 May) describes that by a similar procedure, circadian rhythms of clock genes in keratinocytes were detected by culturing and proliferating keratinocytes adhering to a hair root portion of a hair plucked off from a human subject.

The biological clock estimation method disclosed in PCT Patent Publication No. WO2004/012128 is a method based on expression levels of mRNA in a standard sample collected from an individual organism. In PCT Patent Publication No. WO2004/012128, however, no specific collected tissue (or cells) or assay target gene is described. Because circadian rhythms of clock genes can also be detected in peripheral tissues of the heart, lungs, liver, stomach, spleen, kidneys, testicles and the like (see BMC Molecular Biology 5:18 (2004 Oct. 9)), a method that checks expression levels of clock genes in their peripheral tissues may also be contemplated. However, the collection of these tissues is high in invasiveness to tested individuals so that such a method is not considered to be practical when its application to human subjects is taken into view.

Accordingly, the method that checks expression levels of clock genes in leukocytes collected by blood collection has been conventionally adopted. With this method, however, physical affliction is also entailed upon blood collection. Moreover, an analysis of gene expressions over time requires to collect blood at intervals of several hours and hence to force each human subject to stay in a medical institution. For an assayer, on the other hand, there are problems that time and labor are needed for the isolation of leukocytes from collected blood and the assayer is exposed to a risk of infection from blood. In addition, there is also a potential problem that in the course of the isolation work, the expression of genes in leukocytes may vary due to stimulations caused as a result of the work, thereby possibly making it difficult to perform an accurate assay of expression levels.

As a method that may replace the method making use of leukocytes, a method that uses more-readily collectable skin or tunica mucosa oris may be contemplated (see American Journal of Pathology 158(5):1793-801 (2001 May)). When skin is used, however, punching of cutaneous tissues under topical anesthesia is needed so that this method is not necessarily considered to be a low-invasive method.

On the other hand, the method that uses tunica mucosa oris can limit invasiveness to an extremely low level. Tunica mucosa oris has already been used also in genotyping that uses a genome extracted from tunica mucosa oris. When it is desired to extract mRNA for the purpose of an analysis of a gene expression, however, a problem arises in that mRNA is prone to degradation.

Upon extracting mRNA from a biological tissue, an operation is generally needed to prevent degradation of mRNA. When it is desired to extract mRNA from tunica mucosa oris, however, there is a problem that, even if the above-described operation is conducted, the degradation of mRNA tends to take place due to an RNA-degrading enzyme (RNase) abundantly existing in saliva and the assay results available in the quantification of a gene expression become very unstable.

A primary object of the present invention is, therefore, to newly provide a simple and low-invasive method for obtaining information on a biorhythm of an individual organism.

### DISCLOSURE OF THE INVENTION

To achieve the above-described object, the present invention provides a method for obtaining information on a biorhythm of an individual organism based on variations over time in an expression level of a clock gene in a hair follicle cell of the individual organism.

Examples of the clock gene as a target for the quantification of the expression can include the following genes or homologs thereof (1) to (7):

(1) Per3 gene described in Sequence ID NO: 1 or a homolog thereof,
(2) Per2 gene described in Sequence ID NO: 3 or a homolog thereof,
(3) Bmal1 gene described in Sequence ID NO: 5 or a homolog thereof,
(4) Npas2 gene described in Sequence ID NO: 7 or a homolog thereof,
(5) Nr1d1 gene described in Sequence ID NO: 9 or a homolog thereof,
(6) Nr1d2 gene described in Sequence ID NO: 11 or a homolog thereof, and
(7) Dbp gene described in Sequence ID NO: 13 or a homolog thereof.

In this method, an expression level fluctuation curve indicating the variations over time in the expression level can be used as a molecular clock table for the estimation of the biorhythm.

A phase shift of the biorhythm of the individual organism can be detected by preparing this molecular clock table a plurality of times about the individual organism and collating the resulting molecular clock tables with each other. By collating an expression level of the clock gene at a predetermined time with the molecular clock table, a phase shift of the biorhythm of the individual organism can also be detected.

The term “hair follicle cell” as used herein widely encompasses therein a class of cells adhering to a hair root portion of a body hair drawn out when the body hair is plucked off. More specifically, cells forming an inner root sheath, outer root sheath and papilla are included. It is to be noted that no particular limitation is imposed on the region of a body hair to be plucked off for obtaining hair follicle cells and that, when the present invention is applied to a human subject, for example, a hair, beard, arm hair, leg hair or the like can be used.

Further, the term “individual organism,” which is a target in the present invention for obtaining information on a biorhythm, widely includes mice, rats, monkeys and the like in addition to human subjects, and no particular limitation is imposed thereon insofar as the individual organism is an animal having body hair. When an animal other than human is chosen as a target organism, a target animal homolog of the human clock gene described in Sequence ID NO: 1, 3, 5, 7, 9, 11 or 13 is used.

By the present invention, a method for obtaining information on a biorhythm of an individual organism in a simple and low-invasive manner can be provided.

### BRIEF DESCRIPTION OF THE DRAWINGS

FIG. 1 diagrammatically illustrates by way of example variations over time in the expression level of a clock gene.

FIG. 2 diagrammatically illustrates phase variations of the expression level fluctuation curves of the clock gene.

FIG. 3 diagrammatically illustrates variations over time in the expression levels of clock genes in hair follicle cells (twenty hairs). (A) Per3 gene, (B) Nrld1 gene, (C) Dbp gene, and (D) Nrld2 gene.

FIG. 4 diagrammatically illustrates variations over time in the expression levels of clock genes in hair follicle cells (ten hairs).

FIG. 5 diagrammatically illustrates variations over time in the expression levels of Per3 gene in hair follicle cells (five hairs).

FIG. 6 diagrammatically illustrates variations over time in the expression levels of clock genes in hair follicle cells (beards). (A) five beards, and (B) three beards.

FIG. 7 diagrammatically illustrates variations over time in the expression levels of clock genes in hair follicle cells (twenty arm hairs).

### BEST MODES FOR CARRYING OUT THE INVENTION

To establish a method for obtaining information on a biorhythm of an individual organism in a simple and low-invasive manner, the present inventors focused attention on hair follicle cells as a biological tissue collectable with extremely-low invasiveness.

Like tunica mucosa oris, hair follicle cells are employed in genotyping or the like that uses a genome. However, no attempt has heretofore been made to detect an expression of a clock gene directly from hair follicle cells adhering to a hair root portion of a plucked body hair. As its reason, it was once considered that hair follicle cells adhering to a hair root portion were too small in number to acquire RNA in any amount sufficient for the analysis of a gene expression. As another reason, it was considered difficult to extract RNA with quality sufficient for the analysis of an expression due to an RNA-degrading enzyme (RNase) abundantly existing in sweat.

For the resolution of such quantitative and qualitative problems of RNA, it may be contemplated to conduct an analysis of an expression of a clock gene in hair follicle cells after once culturing and proliferating hair follicle cells adhering to a hair root portion of a plucked hair as described in the above-described Non Patent Document 4.

As a peripheral clock is also found in each individual cell, several circadian rhythm cycles can still be detected even in such cultured hair follicle cells. However, the circadian rhythms so detected are rhythms produced by the hair follicle cells in the state that they had been cultured subsequent to their isolation from an organism and do not reflect a biorhythm synchronized and unified throughout the organism, because as already described, a biorhythm is expressed and regulated as a result of synchronization and unification of a central clock and peripheral clocks in an organism.

The present inventors, therefore, tried to detect a biorhythm by conducting an analysis of an expression of a clock gene directly with respect to a hair follicle cell adhering to a hair root portion of a plucked body hair. As a result, despite such a concern about the quantity and quality of RNA as described above, the present inventors succeeded in detecting an expression level of a clock gene in the hair follicle cell, and further, newly found that the expression level of the clock gene in the hair follicle cell shows a circadian rhythm, leading to the completion of the present invention.

Described specifically, the present invention relates to a method for obtaining information on a biorhythm of an individual organism based on variations over time in the expression level of a clock gene in a hair follicle cell of the individual organism.

In the present invention, no particular limitation is imposed on the collection region of hair follicle cells and, when the present invention is applied to human subjects, for example, hairs, beards, arm or leg hairs, or the like can be used. When the quantification of a gene expression is to be conducted over time, the collection of hair follicle cells is conducted a plurality of time at intervals of several hours. In doing so, it is desired to conduct the respective collections in regions as close as possible to each other to reduce variations in assay conditions.

The number of body hairs to be plucked off in each collection can be, in the case of human subjects, from 5 to 10 for hairs, from 3 to 5 for beards, and from 10 to 20 for arm hairs. The use of hairs, beards or arm hairs as many as the above-mentioned corresponding number or greater makes it possible to extract RNA in an amount sufficient for the quantification of an expression of a clock gene.

The extraction of RNA from hair follicle cells can be conducted by a method known to date, and for example, a commercially-available, RNA extraction kit for trace samples can be used. A reverse transcription reaction (cDNA synthesis) of the extracted RNA can also be conducted by a method known to date, and a commercially-available reverse transcriptase can be used. For the quantification of a gene expression, a real-time PCR assay which permits high-accuracy quantification is in common use at present. In the present invention, this real time PCR assay can also be suitably adopted, and further, various gene expression quantification methods can also be adopted.

Because hair follicle cells which can be collected in a simple and low-invasive manner are used in the method according to the present invention as described above, the physical load on each human subject can be extremely lightened. Different from the existing method that uses leukocytes, the method of the present invention does not require any cumbersome operation so that the results can be obtained in a short time. According to the method of the present invention, the expression level of a gene, therefore, does not vary in the course of a treatment operation of a sample.

As hair follicle cells not subjected to a culturing step are used in the method according to the present invention, the expression level of a clock gene in each hair follicle cell directly reflects the biorhythm of the tested individual at the time of plucking of the body hair. It is, therefore, possible to accurately estimate the biorhythm of the tested individual by conducting the collection of hair follicle cells over time and assaying variations in expression level.

In the present invention, the clock gene as a target of quantification of an expression can be any one of a class of clock genes identified to date. Representative clock genes include Per3 gene (see SEQ ID NOS: 1 and 2), Pert gene (see SEQ ID NOS: 3 and 4), Bmal1 gene (see SEQ ID NOS: 5 and 6), Npas2 gene (see SEQ ID NOS: 7 and 8), Nrld1 gene (see SEQ ID NOS: 9 and 10), Nr1d2 gene (see SEQ ID NOS: 11 and 12), Dbp gene (see SEQ ID NOS: 13 and 14), and the like.

It has become evident that among these, particularly Per3 gene undergoes largest daily fluctuations in expression level in a hair follicle cell and allows to assay variations over time in expression level more clearly compared with the other genes. The biorhythm of a tested individual can, therefore, be estimated more accurately provided that the method is conducted based on variations over time in the expression level of Per3 gene.

Plural ones of the above-described clock genes may be chosen as assay targets. A more accurate estimation is feasible by conducting the estimation of a biorhythm of a tested individual based on the expression levels of plural clock genes showing different daily fluctuation patterns.

It is to be noted that representative clock genes include Per1 gene in addition to the above-described genes. With respect to Per1 gene, however, it has been revealed, by an experiment using mice, that its expression level fluctuates by stress loads (see J. Biol Chem 280(51):42036-43 (2005 Dec. 23, Epub 2005 Oct. 24)). As an unexpected variation in expression level may hence occur under a stress to a tested individual at the time of plucking of a body hair, Per1 gene is considered to be unsuitable as an assay target gene in the present invention.

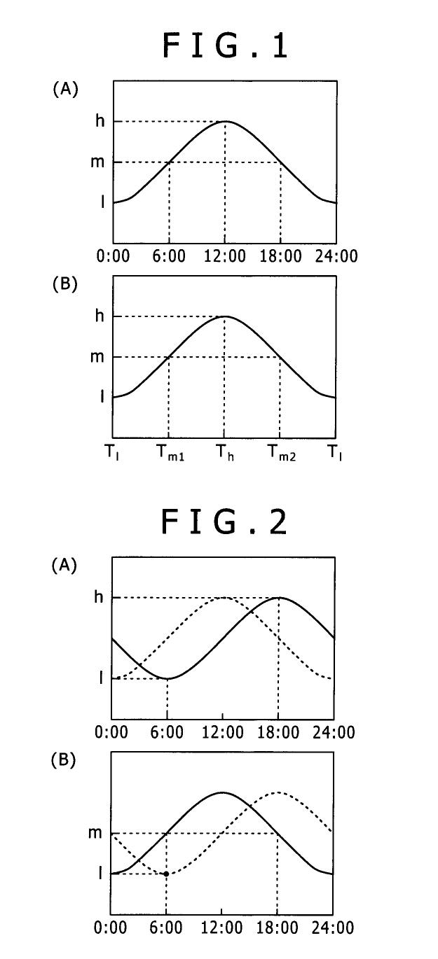

FIG. 1 diagrammatically illustrates variations over time in an expression level of a clock gene in a hair follicle cell. Each diagram shows by way of example an expression level fluctuation curve obtained by plotting expression levels of the clock gene measured by the above-mentioned method at predetermined times in a day. In each diagram, the abscissa represents time, while the ordinate represents expression level.

An expression level fluctuation curve can be obtained by visual inspection from plots of expression levels. To obtain a more accurate curve, a period calculation method such as the auto-correlation method) (correlogram), the power spectrum method, the cosinor method or the periodogram method can also be used.

FIG. 1(A) illustrates by way of example a case in which the expression level was assayed as a low value (l) at 0:00 and as a highest value (h) at 12:00. Ordinary clock times such as “0:00” and “12:00” will hereinafter be called “objective clock times.”

As illustrated in FIG. 1(B), on the other hand, clock times at which the expression level of the clock gene in this tested individual reaches a lowest value (l), a highest value (h) and an intermediate value (m) are represented by “Tₗ,” “Tₕ” and “Tₘ₁ or Tₘ₂,” respectively. These Tₗ, Tₕ and Tₘ will be defined as “subjective clock times” in the sense that “they are clock times determined based on variations over time in the expression level of the clock gene in the tested individual.” The expression level of a clock gene shows variations over time, which are inherent to each tested individual. These subjective times are, therefore, clock times inherent to each tested individual.

As shown in these diagrams, the expression level of a clock gene undergoes daily fluctuations, and shows a circadian rhythm. It is, therefore, possible to obtain information on a biorhythm of a tested individual based on variations over time in the expression level of a clock gene. Using, as a “molecular clock table,” an expression level fluctuation curve which indicates variations over time in the expression level of a clock gene, a description will hereinafter be made about a method for estimating a biorhythm of a tested individual.

Now assume, for example, that, with respect to a tested individual who is known to have the expression level fluctuation curves (which will hereinafter be used synonymously with “molecular clock tables”) of FIG. 1, the expression level assayed at a predetermined clock time is h. In this case, based on the expression level fluctuation curves (molecular clock tables) of FIGS. 1(A) and 1(B), the circadian rhythm of the tested individual can be estimated to be at 12:00 in terms of objective clock time and at Tₕ in terms of subjective clock time. When the activity level is l, the circadian rhythm of the tested individual can be estimated to be at 0:00 in terms of objective clock time and at Tₗ in terms of subjective clock time. Further, when the activity level is m, the circadian rhythm can be estimated to be at 6:00 or 18:00 in terms of objective clock time and at Tₘ₁ or Tₘ₂ in terms of subjective clock time.

Next assume that with respect to the same tested individual, the expression levels obtained by conducting an assay twice, for example, at an interval of 3 hours are p and q, respectively. If q is higher than p (p<q) at that time, the circadian rhythm of the tested individual can be estimated to be between 0:00 and 12:00, in terms of objective clock time, during which the expression level is in an upward phase. In terms of subjective clock times, on the other hand, the circadian rhythm can be estimated to be between Tₗ and Tₕ. If q is lower than p (q<p), on the other hand, the circadian rhythm of the tested individual can be estimated to be between 12:00 and 24:00, in terms of objective clock time, and between Tₕ and Tₗ, in terms of subjective clock time, during both of which the expression level is in a downward phase. In addition, the objective and subjective clock times in the circadian rhythm can be more accurately estimated by determining a coefficient of fluctuation between p and q (q/p) and correlating it with the gradient of a tangential line to the expression level fluctuation curve.

As described above, it is possible to set an optimal dosage time, activity time or meal ingestion time by finding out subjective clock times of a tested individual based on an expression level fluctuation curve (molecular clock table) which indicates variations over time of the expression level of a clock gene. As mentioned above, a biorhythm widely governs biological responses (physiological processes) such as sleep-wake cycles, body temperature, blood pressure, hormonal secretions, mental and physical activities, locomotor ability, drug sensitivity, and the like. Therefore, optimal bedtime and wake-up time, dosage time, activity time, meal ingestion time and the like can be set when such physiological processes are estimated based on subjective clock times.

By finding out objective and subjective clock times based on a molecular clock table, it is also possible to detect a shift in the biorhythm of a tested individual if such a shift arises. A specific description will hereinafter be made about a method for detecting a shift in the biorhythm of a tested individual.

An expression level fluctuation curve (molecular clock table) can be characterized in profile by its highest value (or lowest value), the observation clock time of the highest value (or lowest value), an inclination from the highest value to the lowest value (or from the lowest value to the highest value), and the like. In the present invention, this profile of the expression level fluctuation curve (molecular clock table) will be called “phase.” The profile of a circadian rhythm of a tested individual as specified by an expression level fluctuation curve (molecular clock table) will also be called “phase.”

A biorhythm shift can be detected based on a variation in the phase of the expression level fluctuation curve (molecular clock table).

Described specifically, the expression level fluctuation curve (molecular clock table) illustrated in FIG. 1(A) has a profile, in other words, “a phase” characterized by the lowest value 1, the highest value h, the objective clock time 0:00 of the lowest value, and the objective clock time 12:00 of the highest value.

FIG. 2 diagrammatically illustrates phase variations of expression level fluctuation curves. In FIG. 2(A), the expression level fluctuation curve represented by a dotted line is the curve (which may hereinafter be also called “the molecular clock table 1”) shown in FIG. 1(A), while a solid line represents an illustrative expression level fluctuation curve (which may hereinafter be also called “the molecular clock table 2”) obtained by conducting a similar assay as in FIG. 1(A) on a different assay date. In each diagram, the abscissa represents time, while the ordinate represents expression level.

The molecular clock table 1 has a phase in which the objective clock time corresponding to a lowest value (l) is 0:00 and the objective clock time corresponding to a highest value (h) is 12:00. In the molecular clock table 2, on the other hand, the objective clock time corresponding to a lowest value (l) is 6:00 and the objective clock time corresponding to a highest value (h) is 18:00, and therefore, its phase has varied. This can also be taken as variations in the subjective clock times (Tₗ, Tₕ) in the tested individual.

Described specifically, a shift took place in the phase of an activity fluctuation curve of the tested individual between the time point of the preparation of the molecular clock table 1 and the time point of the preparation of the molecular clock table 2, so that the biorhythm of the tested individual at the time point of the preparation of the molecular clock table 2 was delayed by 6 hours (or advanced by 18 hours) in terms of objective clock time from the time point of the preparation of the molecular clock table 1. As described above, it is possible to detect a shift in the phase of the biorhythm of a tested individual by preparing a molecular clock table a plurality of times with respect to the same tested individual and correlating the resulting molecular clock tables with each other.

As an alternative method for detecting a shift in the phase of a biorhythm, a method such as that to be described below can be contemplated.

FIG. 2(B) is a diagram illustrating the molecular clock table 1 (also see FIG. 1) and molecular clock table 2 of FIG. 2(A) by changing the former from the dotted line to a solid line and the latter from the solid line to a dotted line.

As described above, when the activity value assayed at a predetermined clock time is m with respect to a tested individual who is known to have the expression level fluctuation curve (molecular clock table) of FIG. 1(A), the circadian rhythm of the tested individual can be estimated to be at 6:00 or 18:00 in terms of objective clock time based on the molecular clock table 1.

Now assume that with respect to the same tested individual, the activity value obtained by conducting an assay at 6:00 on a different assay date changed from m to l (see the circle dot in FIG. 2(B)). In this case, it can be estimated that the circadian rhythm of the tested individual was delayed by 6 hours (or advanced by 18 hours) in terms of objective clock time and varied to the circadian rhythm shown in the molecular clock table 2. This can also be taken as a variation in the subjective clock time (Tₗ) of the tested individual.

A phase shift in the biorhythm of a tested individual can also be more simply detected by correlating the activity value at a predetermined clock table with a molecular clock table, which has been prepared beforehand, as described above.

As has been described above, the method according to the present invention makes it possible to estimate a biorhythm inherent to each tested individual and to detect a shift in the biorhythm by using, as a molecular clock table, an expression level fluctuation curve which indicates variations over time in the expression level of a clock gene.

Therefore, the method according to the present invention can be used for the prevention of various diseases caused by disorders (shifts) in biorhythm and also for the diagnosis and treatment of poor physical conditions such as time lag. If correlations can be revealed between the subjective times of a tested individual and various indices indicating physiological processes such as body temperature, blood temperature and hormonal secretions, these indices can be measured based on the subjective clock times, and can then be used for the diagnosis and treatment of diseases caused by disorders in the biorhythm.

In the following examples, specific assay data of expression levels of clock genes in hair follicle cells will be given.

### EXAMPLES

### Example 1

### Expression Quantification 1 of Clock Genes by Use of Hairs

In Example 1, the quantification of expression levels was conducted with respect to clock genes in follicle cells of hairs. Twenty hairs were used in this example.

Hairs were plucked off from three human subjects (human subjects K, T, A) to obtain hair follicle cells. The collection of the hair follicle cells was conducted 9 times at 11:00, 14:00, 17:00, 20:00, 23:00, 2:00, 5:00, 8:00 and 11:00. Using a commercially-available, RNA extraction kit (“RNAeasy micro kit,” Qiagen K.K.), total RNA was extracted from the hair follicle cells in accordance with an accompanying protocol. After a reverse transcription reaction (“Ribatora ace,” Toyobo Co., Ltd.) was conducted in a usual way, gene expression levels were assayed with respect to Per3 gene, Nrdld1 gene, Nr1d2 gene and Dbp gene by using a real-time PCR system (“Prism 7000,” Applied Biosystems). The sequences of primers used for the respective genes are shown in Table 1.

TABLE 1

| | | SEQ | | SEQ |
|---|---|---|---|---|
| | | ID | | ID |
| Gene | Forward primer | NO | Reverse primer | NO |
| Per3 | CTACCTGCACCCTGAAGATCGTTCTC | 15 | CTGGAATCCAGTATGATGTAGTCTCCGTTT | 16 |
| Per2 | GCTTCCCTTGTGGAGCATGT | 17 | TTGACCAGGTAGGGCGTCAT | 18 |
| Bma11 | CATGCAACGCAATGTCCAGGAAATTAGATA | 19 | CCTGAGAATGAGGTGTTTCAATTCATCGTC | 20 |
| Npas2 | CGGGACCAGTTCAATGTTCTCATCAAAGAG | 21 | GTCTTGCTGAATGTCACAGATTTCCGTTT | 22 |
| Nrld1 | GCTCAGTGCCATGTTCGACTTC | 23 | AAGTCTCCAAGGGCCGGTTC | 24 |
| Nrld2 | TCCAGTACAAGAAGTGCCTGAAGAATGAAA | 25 | CACGCTTAGGAATACGACCAAACCGA | 26 |
| Dbp | TCGTTGTTCTTGTACCGCCGGC | 27 | CCAGCTGATCTTGCCCTATCAAGCATTC | 28 |
| 18s | CGCCGCTAGAGGTGAAATTC | 29 | CGAACCTCCGACTTTCGTTCT | 30 |
| rRNA | | | | |

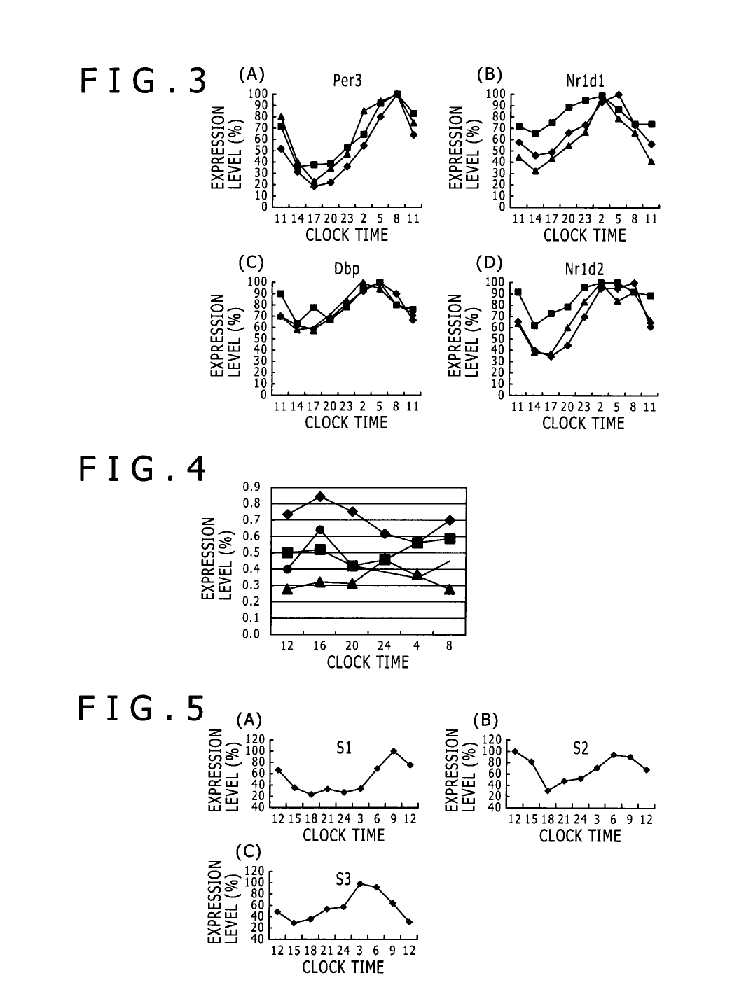

The results are shown in FIG. 3. FIG. 3(A) shows the results on Per3 gene, FIG. 3(B) illustrates the results on NRdld1 gene, FIG. 3(C) depicts the results on Dbp gene, and FIG. 4(D) shows the results on Nrld2 gene. In each diagram, the plots indicated by rhombus dots represent the results on the human subject K, square dots represent the results on the human subject T, and triangle dots represent the results on the human subject A. In each diagram, the abscissa represents sampling clock time, while the ordinate represents gene expression level. It is to be noted that each gene expression level was subjected to an expression level correction by using 18s rRNA as an internal standard and was then indicated as a relative value while assuming that the highest value of the corresponding gene expression level in the corresponding human subject was 100.

The Per3 gene expression level in the human subject K (see the rhombus dots in FIG. 3(A)), after initiation of sampling at 11:00, decreased at 14:00 and showed a lowest value at 17:00. Subsequently, the Per3 gene expression level increased through 20:00, 23:00, 2:00 and 5:00, and showed a highest value at 8:00. Further, the Per3 gene expression level again decreased at 11:00, and showed a similar expression level as observed at the time point of the initial sampling (11:00).

The Per3 gene expression level in the human subject T (see the square dots in FIG. 3(A)), after initiation of sampling at 11:00, showed a lowest value at 14:00 and turned into a slight increase at 17:00. Subsequently, the Per3 gene expression level increased through 20:00, 23:00, 2:00 and 5:00, and a highest value was observed at 8:00. The Per3 gene expression level was similar to that in the human subject K in that it again decreased at 11:00 and showed a similar expression level as observed at the time point of the initial sampling (11:00). Further, the Per3 gene expression level in the human subject A (see the triangle dots in FIG. 3(A)) showed substantially similar variations over time as that in the human subject A.

From the foregoing, it has been indicated that the circadian rhythm of Per3 gene can be detected by assaying daily fluctuations of Per3 gene based on a real-time PCR assay that uses hair follicle cells of twenty hairs. As the detected circadian rhythm of Per3 gene showed a characteristic phase in each human subject, it has been indicated that a biorhythm inherent to each human subject can be detected by the method according to the present invention.

With respect to each of Nrdld1 gene, Dbp gene and Nrld2 gene shown in FIGS. 3(B) to 3(D), it was possible to detect daily fluctuations in expression level and a circadian rhythm, and therefore, to detect a biorhythm inherent to each human subject.

Paying attention to the lowest value of the daily fluctuation on each gene, the lowest value was approximately 55% of the highest value in the case of Dbp gene (C) and was approximately 35% of the highest value in the case of each of Nrdldl gene (B) and Nrld2 gene (D), while the lowest value in the case of Per3 gene (A) was approximately 20% of the highest value, and therefore, was pronouncedly lower compared with those for the other genes. Namely, Per3 gene can be considered to be greater in the range of daily fluctuations compared with the other genes and to permit clearly detecting variations in expression level. It has, therefore, been revealed that the use of Per3 gene makes it possible to more accurately estimate a biorhythm.

### Example 2

### Expression Quantification 2 of Clock Genes by Use of Hairs

In Example 2, the quantification of expression levels was conducted with respect to clock genes in follicle cells of hairs. A half of the hairs in Example 1, ten hairs, were used in this example.

Hairs were plucked off from one human subject (human subject B) to obtain hair follicle cells. The collection of the hair follicle cells was conducted 6 times at 12:00, 16:00, 20:00, 24:00, 4:00, and 8:00. In a similar manner as in Example 1, gene expression levels were assayed with respect to Per2 gene, Bmal1 gene, Npas2 gene and Nrd1d2 gene.

The results are shown in FIG. 4. In the diagram, the plots indicated by square dots represent the results on Per2 gene, rhombus dots represent the results on Bmal1 gene, circle dots represent the results on Npas2 gene, and triangle dots represent the results on Nrdld2 gene. In the diagram, the abscissa represents sampling clock time, while the ordinate represents gene expression level. It is to be noted that each gene expression level was indicated as a relative value while assuming that the expression level of 18s rRNA used as an internal standard was 1.

As shown in the diagram, it has been revealed that daily fluctuations and circadian rhythm of each gene can be detected even when the number of hairs to be used is decreased to 10.

### Example 3

### Expression Quantification 3 of Clock Genes by Use of Hairs

In Example 3, the number of hairs to be used was decreased further to 5, and the quantification of expression levels of clock genes was conducted.

Hairs were plucked off from three human subjects (human subjects S1, S2, S3) to obtain hair follicle cells. The collection of the hair follicle cells was conducted 9 times at 12:00, 15:00, 18:00, 21:00, 24:00, 3:00, 6:00, 9:00 and 12:00. In a similar manner as in Example 1, gene expression levels of Per3 gene were assayed.

The results are shown in FIG. 5. FIG. 5(A) shows the results on the human subject S1, FIG. 5(B) illustrates the results on the human subject S2, and FIG. 5(C) depicts the results on the human subject S3. In each diagram, the abscissa represents sampling clock time, while the ordinate represents gene expression level. It is to be noted that each gene expression level was subjected to an expression level correction by using 18s rRNA as an internal standard and was then indicated as a relative value while assuming that the highest value of the corresponding expression level in the corresponding human subject was 100.

As shown in the diagrams, it has been revealed that, even when the number of hairs to be used is decreased to 5, daily fluctuations and circadian rhythm of Per3 gene can be detected and a biorhythm inherent to each human subject can be detected.

### Example 4

### Expression Quantification 1 of Clock Genes by Use of Body Hairs

In Example 4, the quantification of expression levels of clock genes in hair follicle cells was conducted by using beards. Five or three beards were used in this example.

Beards were plucked off from two human subjects (human subjects C, D) to obtain hair follicle cells. The collection of the hair follicle cells was conducted 7 times at 12:00, 16:00, 20:00, 24:00, 4:00, 8:00, and 12:00. In a similar manner as in Example 1, gene expression levels were assayed with respect to Per3 gene, Bmal1 gene, Nrdld1 gene, Nrld2 gene and Dbp gene.

The results are shown in FIG. 6. FIG. 6(A) shows the results of an assay conducted by using five beards plucked off from the human subject C, and FIG. 6(B) illustrates the results of an assay conducted by using three beards plucked off from the human subject D. In each diagram, the plots indicated by squares represent the results on Per3 gene, rhombus dots represent the results on Bmal1 gene, inverted triangle dots represent the results on Nrd1d1 gene, triangle dots represent the results on Nrd1d2 gene, and circles represent the results on Dbp gene. In each diagram, the abscissa represents sampling clock time, while the ordinate represents gene expression level. It is to be noted that each gene expression level was subjected to an expression level correction by using 18s rRNA as an internal standard and was then indicated as a relative value while assuming that the highest value of the expression level of the corresponding gene in the corresponding human subject was 1.

As shown in the diagrams, it has been revealed that, when beards are used in place of hairs, daily fluctuations and circadian rhythm of each gene can also be detected and a biorhythm inherent to each human subject can also be detected. With respect to beards, the use of three beards permitted the detection of a biorhythm.

Concerning Per3 gene, a circadian rhythm with a greater amplitude than those of the other genes was recognized as in the case of hairs.

### Example 5

### Expression Quantification 3 of Clock Genes by Use of Body Hairs

In Example 5, the quantification of expression levels of clock genes in hair follicle cells was conducted by using arm hairs (between the wrist to the elbow). Twenty arm hairs were used in this example.

Arm hairs were plucked off from one human subject (human subject E) to obtain hair follicle cells. The collection of the hair follicle cells was conducted 6 times at 18:00, 22:00, 2:00, 6:00, 10:00, and 14:00. In a similar manner as in Example 1, gene expression levels were assayed with respect to Per2 gene and Bmal1 gene.

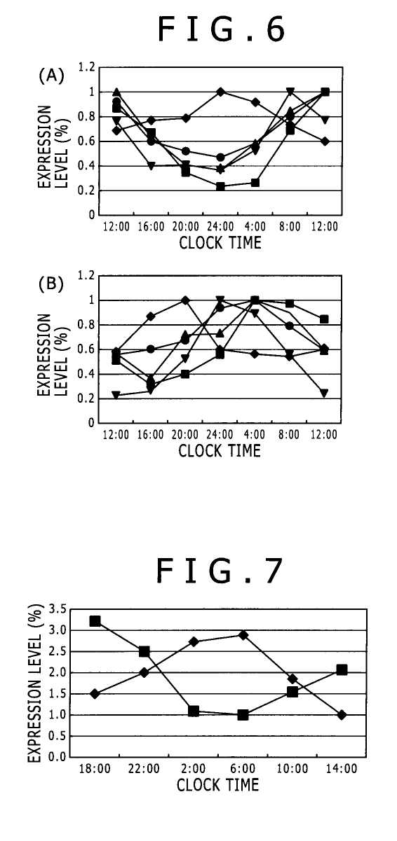

The results are shown in FIG. 7. In the diagram, the plots indicated by square dots represent the results on Per2 gene, and rhombus dots represent the results on Bmal1 gene. In the diagram, the abscissa represents sampling clock time, while the ordinate represents gene expression level. It is to be noted that each gene expression level was subjected to an expression level correction by using 18s rRNA as an internal standard and was then indicated as a relative value while assuming that the lowest value of the expression level of the corresponding gene was 1.

As shown in the diagram, it has been revealed that, when arm hairs are used in place of hairs, daily fluctuations and circadian rhythm of each gene can be detected. With respect to arm hairs, the use of twenty arm hairs permitted the detection of a biorhythm.

### INDUSTRIAL APPLICABILITY

The method according to the present invention makes it possible to estimate a biorhythm inherent to each tested individual in a simple and low-invasive manner by using, as a molecular clock table, an expression level fluctuation curve which indicates variations over time in the expression level of a clock gene in a follicle cell. It is, therefore, possible for each individual to find out a biorhythm inherent to himself or herself and to set an optimal dosage time, activity time and/or meal ingestion. The method according to the present invention can thus be used for the realization of chronotherapy, the exhibition of one's own ability, and/or dieting.

Further, the detection of a shift in the phase of a biorhythm by the method according to the present invention is useful for the prevention of various diseases caused by the biorhythm shift and also for the improvement of poor physical conditions such as time lag.

1. A method for obtaining information on a biorhythm of an individual organism based on variations over time in an expression level of a clock gene in a hair follicle cell of the individual organism.

2. The method according to claim 1, wherein the clock gene is at least one gene selected from the following genes or homologs thereof (1) to (7):
(1) Per3 gene described in Sequence ID NO: 1 or a homolog thereof,
(2) Per2 gene described in Sequence ID NO: 3 or a homolog thereof,
(3) Bmal1 gene described in Sequence ID NO: 5 or a homolog thereof,
(4) Npas2 gene described in Sequence ID NO: 7 or a homolog thereof,
(5) Nr1d1 gene described in Sequence ID NO: 9 or a homolog thereof,
(6) Nr1d2 gene described in Sequence ID NO: 11 or a homolog thereof, and
(7) Dbp gene described in Sequence ID NO: 13 or a homolog thereof.

3. The method according to claim 1, wherein an expression level fluctuation curve indicating the variations over time in the expression level is used as a molecular clock table for the estimation of the biorhythm.

4. The method according to claim 3, wherein a phase shift of the biorhythm of the individual organism is detected by collating, with each other, those obtained by preparing the molecular clock table a plurality of times about the individual organism.

5. The method according to claim 3, wherein a phase shift of the biorhythm of the individual organism is detected by collating an expression level of the clock gene at a predetermined time with the molecular clock table.
